(19) United States
(12) Patent Application Publication
(10) Pub. No.: US 2026/0350272 A1
(43) Pub. Date: Nov. 5, 2026

(54) DEVICE AND METHOD FOR DESIGNING CATALYST USING ARTIFICIAL INTELLIGENCE

(71) Applicant: KOREA ADVANCED INSTITUTE OF SCIENCE AND TECHNOLOGY, Daejeon (KR)

(72) Inventors: Jeung Ku KANG, Daejeon (KR); Dong Won KIM, Daejeon (KR); Jong Hui CHOI, Daejeon (KR); Do Hwan JUNG, Daejeon (KR); Ji Hoon KIM, Daejeon (KR)

(21) Appl. No.: 19/530,992

(22) Filed: Feb. 5, 2026

(30) Foreign Application Priority Data
Apr. 21, 2025 (KR) 10-2025-0051762

(51) Int. Cl. G06F 30/27

(52) U.S. Cl. CPC G06F 30/27

(57) ABSTRACT

Embodiments relate to a device and method for designing a catalyst satisfying a high entropy criterion using artificial intelligence. A device according to one embodiment comprises: a dispenser; a measuring instrument; and a control unit that designs experimental conditions and an initial composition for a catalyst, synthesizes multiple catalysts based on the experimental conditions and the initial composition through the dispenser, evaluates multiple overpotentials corresponding to the catalysts to generate a composition-overpotential dataset, derives a final catalyst using the composition-overpotential dataset through a pre-designated artificial intelligence model, and analyzes the physical properties of the final catalyst through the measuring instrument.

### CROSS-REFERENCE TO RELATED APPLICATION

This application claims priority to and the benefit of Korean Patent Application No. 10-2025-0051762, filed on Apr. 21, 2025, the disclosure of which is incorporated herein by reference in its entirety.

### BACKGROUND

The disclosure relates to a device and method for designing multi-principal element catalysts using artificial intelligence (AI).

Hydrogen, the most energy-dense chemical fuel by mass, delivers 142 MJ kg¹—nearly three times the energy density of gasoline. Green hydrogen, produced through water splitting powered by renewable sources such as solar energy, offers an environmentally sustainable alternative to blue hydrogen, thanks to the use of electrocatalysts. The development of materials with low overpotentials is pivotal to advancing green hydrogen production. Alloys with multiple principal elements (MPEAs), such as HEAs (HEAs), could accommodate multiple active sites, offering a promising option for optimizing water-splitting compositions. However, the vast compositional spaces of MPEAs pose a formidable challenge, one beyond the reach of conventional experimental methods. Moreover, affected by a number of variables (e.g., magnetism, phonons, vacancy effect), not all compositions result in a solid solution structure; rather, they can result in numerous types of microstructures such as metallic glasses, intermetallic mixtures, and composites.

One possible solution to address this issue is the use of AI. As the computational power and the efficiency of algorithm evolves, its potential to revolutionize materials discovery becomes increasingly evident. Nevertheless, contemporary deep learning models, high-entropy alloys reliant on data mining through paper scraping or ab initio calculations, often face limitations in terms of utilization of small-sized experimental dataset due to its scaling law and, more severely, transparency of the model. Although there have been some efforts to understand its underlying working principles, deep learning models, sometimes referred to as ‘black-box’ models, oftentimes face limitations in terms of its explainability. It is particularly important for the field of ‘AI for science’, since an explainable AI model would not only provide reasons to trust the results given by AI, but also offer underlying scientific insights. In designing multi-principal element alloys for efficient water splitting, a challenging yet important aspect is mapping overpotential across the compositional space.

For the sake of explainability and credibility of the result given by AI, Gaussian Process Regression (GPR), informed by controlled experimental data, offers a promising solution to the challenge by predicting both expected values and associated uncertainties. Nevertheless, due to the lack of prior knowledge in fields of materials design such as dimensionality constraints and insufficient boundary information, bare GPR model combined with Bayesian Optimization (BO) procedure may lead to convergence on local minima in low-entropy regions, such as composites of binary or ternary alloys. Since standard acquisition functions (e.g., upper confidence bound, expected improvement) do not account for compositional boundaries, in extreme cases, they may even recommend negative composition of a certain element if that element is recognized as undesirable for the target function.

Therefore, a solution to address these issues is needed.

### RELATED ART DOCUMENT

### Patent Document

(Patent document 1) Republic of Korea Patent No. 10-1949448
(Patent document 2) Republic of Korea Patent No. 10-2640009
(Patent document 3) Republic of Korea Publication No. 10-2024-0083839

### SUMMARY

An aspect of the disclosure is to design a catalyst that meets a high-entropy criterion by using artificial intelligence.

Another aspect of the disclosure is to design a high-entropy alloy, which is a catalyst, by using artificial intelligence.

The aspect of the disclosure is not limited to that mentioned above, and other aspects not mentioned will be clearly understood by those skilled in the art from the description below.

A device for designing a catalyst using artificial intelligence, according to the disclosure, includes: a dispenser; a measuring instrument; and a control unit that designs experimental conditions and an initial composition for a catalyst, synthesizes multiple catalysts based on the experimental conditions and the initial composition through the dispenser, evaluates multiple overpotentials corresponding to the catalysts to generate a composition-overpotential dataset, derives a final catalyst using the composition-overpotential dataset through a pre-designated artificial intelligence model, and analyzes the physical properties of the final catalyst through the measuring instrument.

A method for designing a catalyst using artificial intelligence, according to the disclosure, includes: designing, by a control unit, experimental conditions and an initial composition for a catalyst; synthesizing, by the control unit, multiple catalysts based on the experimental conditions and initial composition through a dispenser; evaluating, by the control unit, multiple overpotentials corresponding to the catalysts to generate a composition-overpotential dataset; deriving, by the control unit, a final catalyst using the composition-overpotential dataset through a pre-designated artificial intelligence model; and analyzing, by the control unit, the physical properties of the final catalyst through a measuring instrument.

An effect of the disclosure is to accurately design a catalyst that meets the high-entropy criterion by using artificial intelligence.

Another effect of the disclosure is to rapidly design a high-entropy alloy, which is a catalyst, by using artificial intelligence.

The effects of the disclosure are not limited to the effects described above, and should be understood to include all effects that are inferable from the configuration of the disclosure described in the detailed description or claims of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram of a device for designing a catalyst according to an embodiment of the disclosure;

FIG. 2 is a block diagram of a control unit of a device for designing a catalyst according to an embodiment of the disclosure;

FIG. 3 is a view illustrating a process for designing a catalyst in a device for designing a catalyst according to an embodiment of the disclosure;

FIG. 4 is a graph illustrating the performance of an artificial intelligence model according to an embodiment of the disclosure;

FIG. 5 is a graph illustrating the performance of a design of experiments-based sampling technique according to an embodiment of the disclosure;

FIG. 6 is a view illustrating a process for designing a catalyst using an artificial intelligence model in a device according to an embodiment of the disclosure;

FIG. 7 is a graph illustrating the relationship between overpotential and a high-entropy alloy according to an embodiment of the disclosure;

FIGS. 8A to 8O are graphs illustrating the structural characteristics of a high-entropy alloy according to an embodiment of the disclosure;

FIGS. 9A to 9I are graphs illustrating electrochemical test results of a high-entropy alloy according to an embodiment of the disclosure;

FIG. 10 is a flowchart illustrating a process for searching for a final catalyst in a device according to an embodiment of the disclosure; and

FIG. 11 is a flowchart illustrating a process for designing a catalyst using artificial intelligence in a device according to an embodiment of the disclosure.

### DETAILED DESCRIPTION

Hereinafter, the disclosure will be described with reference to the accompanying drawings. However, the disclosure may be implemented in various different forms and therefore is not limited to the embodiments described herein. In addition, in order to clearly describe the disclosure in the drawings, parts that are not related to the description are omitted, and similar parts are given similar drawing reference numerals throughout the specification.

In the entire specification, when a part is said to be “connected (linked, contacted, coupled)” to another part, this includes not only the case where it is “directly connected” but also the case where it is “indirectly connected” with another member in between. In addition, when a part is said to “include” a component, this does not mean that it excludes other components, unless otherwise specifically stated, but rather that it may include other components.

The terms used in this specification are used only to describe specific embodiments and are not intended to limit the disclosure. The singular expression includes the plural expression unless the context clearly indicates otherwise. In this specification, the terms “include” or “have” are intended to specify the presence of a feature, number, step, operation, component, part, or combination thereof described in the specification, but should be understood as not excluding in advance the possibility of the presence or addition of one or more other features, numbers, steps, operations, components, parts, or combinations thereof.

Hereinafter, embodiments of the disclosure will be described in detail with reference to the accompanying drawings.

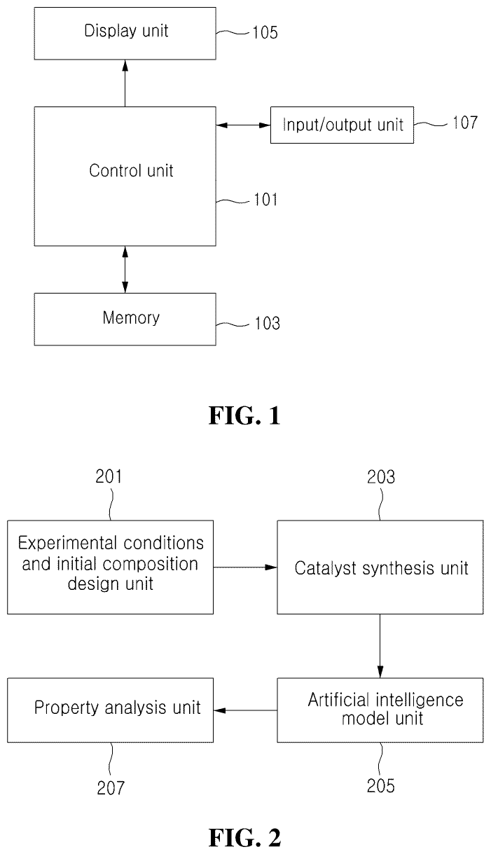

FIG. 1 is a block diagram of a device for designing a catalyst according to an embodiment of the disclosure.

Referring to FIG. 1, the device for designing a catalyst includes a control unit (101), a memory (103), a display unit (105), and an input/output unit (107).

Regarding each component, the memory (103) stores a variety of programs and data required for the operation of the device for designing a catalyst. For example, the memory (103) may be implemented as a non-volatile memory, volatile memory, flash memory, a hard disk drive (HDD), or a solid-state drive (SSD).

The display unit (105) displays image data under the control of the control unit (101). The implementation of the display unit (105) is not limited, and may be implemented as various display types, such as a Liquid Crystal Display (LCD), an Organic Light Emitting Diode (OLED) display, an Active-Matrix Organic Light-Emitting Diode (AMOLED), or a Plasma Display Panel (PDP).

The display unit (105) may include additional components depending on its implementation method. For example, if the display unit (105) is a liquid crystal display (LCD), the display unit (105) may include an LCD display panel (not shown), a backlight unit (not shown) that supplies light to the LCD display panel, and a panel driving board (not shown) that drives the panel. The display unit (105) may be coupled with a touch panel (not shown) of the Input/output unit (107) to provide a touch screen (not shown).

The Input/output unit (107) receives various commands from the user or outputs a variety of data to an external device. For example, the Input/output unit (107) may include at least one of a key, a touch panel, and a pen recognition panel.

Additionally, the device for designing a catalyst may include a communication unit (not shown). The communication unit (not shown) communicates with various types of external devices using various types of wired or wireless communication schemes. For example, the communication unit (not shown) may include a Wi-Fi module and an Long Term Evolution (LTE) module. For example, the Wi-Fi module communicates via Wi-Fi, and the LTE module may communicate according to various communication standards, such as IEEE and LTE.

The control unit (101) controls the overall operation of the device for designing a catalyst using various programs stored in the memory (103).

For example, as illustrated in FIG. 2, the control unit (101) may include an experimental conditions and initial composition design unit (201), a catalyst synthesis unit (203), an artificial intelligence model unit (205), and a property analysis unit (207).

Regarding each component, the experimental conditions and initial composition design unit (201) may design experimental conditions (or synthesis conditions) and initial composition for the catalyst based on a Design of Experiment (DoE).

For example, experimental design may be a method of conducting experiments to efficiently gather information under specific conditions. An experimental design may have two aspects, the selection of experimental treatments (A) and the method of experimental arrangement (B).

For example, the selection of experimental treatments (A) refers to the selection of factors and levels, factors are causal systems that influence the characteristic values of experimental results, and levels may refer to each step taken by a factor. The method of experimental arrangement (B) may be developed based on Fisher's three principles, \textcircled{1} replication, \textcircled{2} randomization, and \textcircled{3} local control. Replication refers to the repetition of experiments under identical conditions, which allows for statistical estimation of error variance. Furthermore, randomizing the experimental order may convert errors into statistically unbiased errors (random errors).

For example, the catalyst, which is used to produce green hydrogen, may be either a low-entropy alloy or a high-entropy alloy. The high-entropy alloy may be a bifunctional catalyst capable of both hydrogen evolution reactions (HER) and oxygen evolution reactions (OER). For example, experimental conditions (or synthesis conditions) refer to the conditions for synthesizing elements to produce a catalyst, and the initial composition may refer to the initial values for the content and ratio of each element when the catalyst is composed of several elements.

At this time, the experimental conditions and initial composition design unit (201) determines the synthesis conditions using a synthesis process optimization technique and designs the initial catalyst based on the determined synthesis conditions.

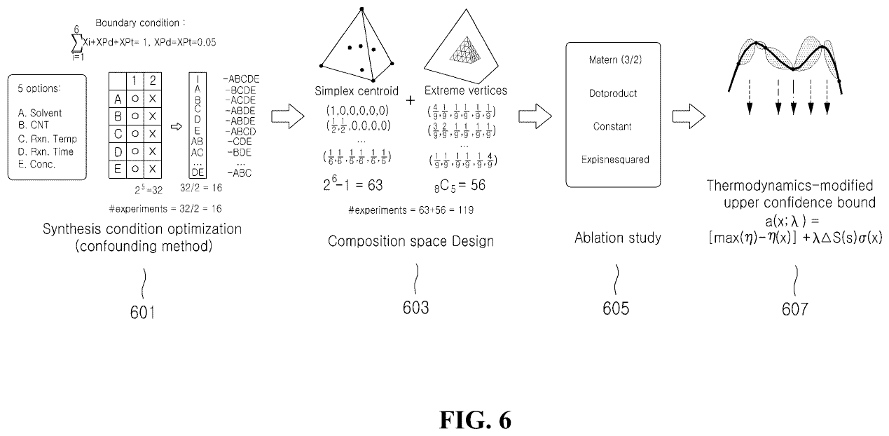

For example, the synthesis process optimization technique (601) may be controlled by five synthesis conditions (e.g., metal precursor concentration, solvent, additive, reaction time, and reaction temperature), as illustrated in FIG. 6. When each synthesis condition is represented by the symbols A, B, C, D, and E, A represents the solvent condition (oleylamine (OAm) versus ethanol), B represents the additive (carbon nanotubes (CNTs) versus no CNTs), C represents the reaction temperature (200 °C versus 120 °C), D represents the reaction time (12 hours versus 2 hours), and E represents the metal precursor concentration (5 mM versus 10 mM). The synthesis process optimization technique (601) may employ a writing scheme where a symbol is indicated when a particular synthesis condition corresponds to the former, and no symbol is indicated when a particular synthesis condition corresponds to the latter. For example, the combination “ACD” could represent the combination of (OAm, no CNT, 200 °C for 12 hours, 10 mM).

In this way, if there is a negative correlation between two options for each synthesis condition, the total number of possible synthetic scenarios (32) may be organized into two groups of 16. For example, I(ethanol, no CNT, 120 °C for 2 hours, 10 mM)=−ABCDE, A=−BCDE, B=−ACDE, . . . , DE=−ABC, etc. Using this organization method, the synthesis process optimization technique (601) may optimize the synthesis process by selecting the highest-performing synthesis process within 16 experiments for five conditions. For example, the optimized synthesis process may include the synthesis conditions: (OAm solvent, CNT use, 200 °C heat treatment, 2 hours of heat treatment, and 5 mM precursor concentration).

The experimental conditions and initial composition design unit (201) may then design the catalyst based on the optimized synthesis process and design the initial composition of the resulting catalyst. For example, the initial composition may be pre-designed using a design of experiments (DoE-based sampling) technique. For example, the DoE-based sampling technique may select features from a dataset to train an artificial intelligence model.

The catalyst synthesis unit (203) can automatically synthesize multiple catalysts based on the designed experimental conditions and initial composition using a dispenser (liquid handler) (not shown). At this time, the catalyst synthesis unit (205) can synthesize multiple catalysts with the same constituent elements but different compositions using a DoE-based sampling technique.

For example, a sampling technique based on a design of experiments (DOE) may be used to train an artificial intelligence model. If the artificial intelligence model's dataset is controlled to have six features (x1, . . . , x6) and one parameter (y), a DOE-based sampling technique can select a total of 63 features, from (1, 0, 0, 0, 0, 0) through (½, ½, 0, 0, 0, 0) to (⅙, ⅙, ⅙, ⅙, ⅙, ⅙). Additionally, the extreme vertices design is applied to sample points with high-entropy region with each element composition set to

$$\frac{1}{9} \leq {x}_{i} \leq \frac{4}{9}$$

and the grid size was set to 1/9 for convenience, resulting in ₈C₃=56 combinations from ( 4/9, 1/9, 1/9, 1/9, 1/9, 1/9), ( 3/9, 2/9, 1/9, 1/9, 1/9, 1/9) to ( 1/9, 1/9, 1/9, 1/9, 1/9, 4/9) That is, through a sampling technique based on a design of experiments, six compositional spaces may be evenly distributed, resulting in the selection of 119 points. For example, the catalyst synthesis unit (203) may automatically synthesize samples corresponding to 119 compositions based on the 119 points selected through the dispenser.

Furthermore, the catalyst synthesis unit (203) may evaluate the overpotentials corresponding to the synthesized samples to generate a composition-overpotential dataset. For example, the catalyst synthesis unit (2013) may measure overpotentials corresponding to synthesized samples and generate a composition-overpotential dataset by converting the measured overpotentials corresponding to the synthesized samples into data. For example, the composition-overpotential dataset may be generated by grouping measured overpotential values corresponding to specific compositions.

The artificial intelligence model unit (205) may derive the final catalyst using the composition-overpotential dataset through an artificial intelligence model. For example, the artificial intelligence model may be a GPR model. For example, the final catalyst may be a high-entropy alloy.

More specifically, the artificial intelligence model unit (205) may perform training and testing on the artificial intelligence model using a sampling technique based on a design of experiments. For example, of the 119 pieces of data included in the composition-overpotential dataset, the artificial intelligence model unit (205) may classify 80% thereof as training data (hereinafter referred to as the “training dataset”) and 20% as testing data (hereinafter referred to as the “test dataset”). The artificial intelligence model unit (205) may then derive results for a plurality of kernels based on the training dataset and test dataset classified through the artificial intelligence model, and among the plurality of kernels, the kernel with the best performance can be selected as the primary kernel for configuring the artificial intelligence model. For example, the primary kernel may be the Matern (3/2) kernel.

The artificial intelligence model unit (205) may then derive candidate catalysts using the composition-overpotential dataset through the trained artificial intelligence model. For example, the candidate catalyst represents a catalyst derived through the trained artificial intelligence model and may represent a candidate for the final catalyst. The final catalyst may be a high-entropy alloy.

The artificial intelligence model unit (205) may then check whether the derived candidate catalyst meets a pre-specified completion criterion. For example, the completion criterion represents the criteria for completing the operation of deriving a catalyst using the trained artificial intelligence model and may include a pre-specified number of convergences for the trained artificial intelligence model. For example, the number of convergences may be 150.

If the derived candidate catalyst meets the completion criterion as a result of the check, the artificial intelligence model unit (205) may select the derived candidate catalyst as the final catalyst.

In contrast, if the derived candidate catalyst does not meet the completion criterion, the artificial intelligence model unit (205) may use an acquisition function based on thermodynamic entropy to designate an exploration location (or data point) for the trained artificial intelligence model.

For example, a strategy of extensively exploring uncertain x to increase the generality of the artificial intelligence model is called exploration, while a strategy of exploring x that exhibits good performance based on the artificial intelligence model's current judgment is called exploitation.

For example, the artificial intelligence model unit (205) may generate an acquisition function to create an artificial intelligence model that finds the lowest overpotential while maintaining confidence (low uncertainty) in all regions of the configuration space. Furthermore, the artificial intelligence model unit (205) may use the generated acquisition function to search for data points for additional learning by the artificial intelligence model and designate the searched data points as development locations.

For example, the acquisition function may be a function that indicates that, since the artificial intelligence model does not know about thermodynamic entropy and composition conditions, the amount of information obtained from sampling edge cases is less than the amount of information obtained in high-entropy regions.

For example, the composition conditions may be expressed as in Equation 1 below.

$${\sum }_{i = 1}^{6} {x}_{i} = 1 \qquad [ Equation 1 ]$$

Here, i may represent a configuration space. That is, Equation 1 indicates that the composition condition requires that the sum of the six configuration spaces be 1.

For example, the acquisition function may be designed using Equation 2, as follows:

a(x;λ)=[max(η_{ows})−η_{ows}(x)]+λS_{config}(x)σ(x)

Here, a(x; λ) represents the acquisition function, and σ(x) is the uncertainty returned by the artificial intelligence model, which can represent the uncertainty of the prediction value at configuration x. A is a hyperparameter for the artificial intelligence model at that point, and may represent a hyperparameter that determines which option the artificial intelligence model prefers. η_{ows}(x) may represent the overall water splitting overpotential at x. S_{config}(x) may represent the compositional entropy at x.

For example, once the acquisition function is designed, the optimal value of x may be obtained through Equation 3, as follows:

x=argmax[a(x;λ)] [Equation 3]

Here, x represents the composition that maximizes the acquisition function, and a(x; X) may represent an acquisition function with X as a hyperparameter.

The artificial intelligence model unit (205) may iteratively perform operations such as re-deriving candidate catalysts based on the designated exploration locations using the trained artificial intelligence model and checking whether the derived candidate catalysts meet the completion conditions. For example, the trained artificial intelligence model may perform learning based on the designated exploration locations to derive candidate catalysts.

The property analysis unit (207) may analyze the properties of the derived final catalyst. For example, the property analysis unit (207) may measure the electrochemical properties of the final catalyst using a measuring instrument and store the measured values.

With this configuration, an embodiment of the disclosure may accurately design a catalyst that satisfies the high-entropy criterion using AI. An embodiment of the disclosure may rapidly design a high-entropy alloy as a catalyst using AI.

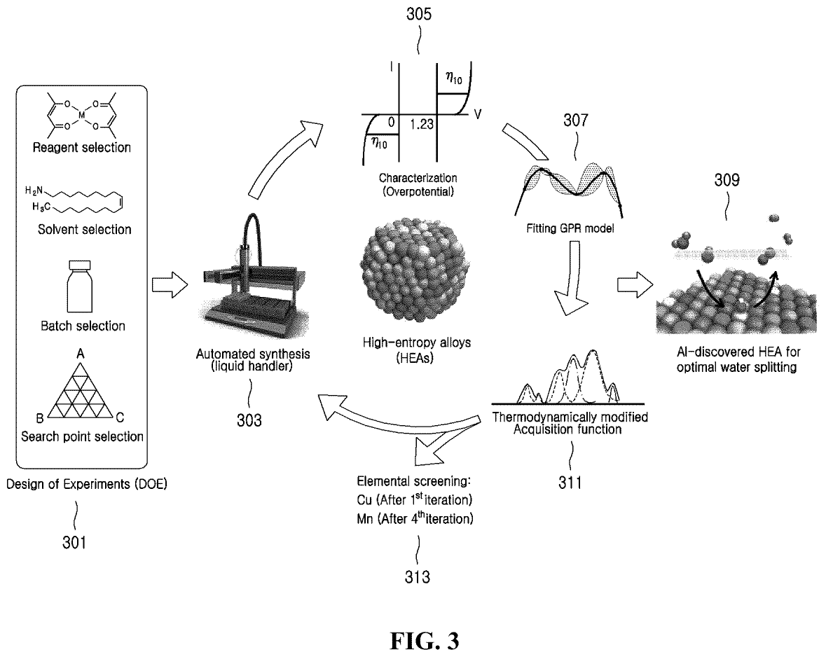

FIG. 3 is a view illustrating a process for designing a catalyst in a device for designing a catalyst according to an embodiment of the disclosure.

Referring to FIG. 3, the process for designing a catalyst includes conducting an experimental design (301), automating synthesis using a dispenser (303), evaluating the catalyst characteristic, overpotential (305), and updating the artificial intelligence model by training and testing it (307). The process for designing a catalyst may also include designing an acquisition function based on thermodynamic entropy (311), deriving a high-entropy alloy based on the designed acquisition function (309), and screening elements included in the derived high-entropy alloy (313).

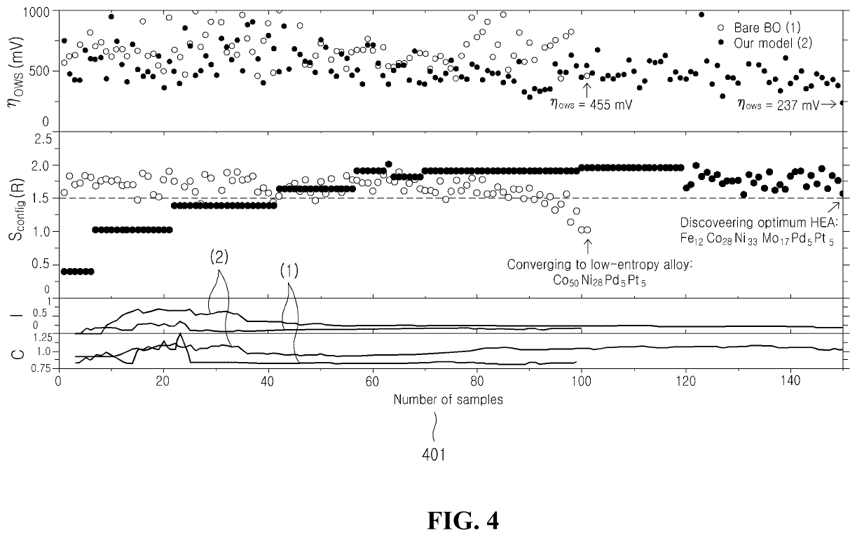

FIG. 4 is a graph illustrating the performance of an artificial intelligence model according to an embodiment of the disclosure.

The graph (401) depicted in FIG. 4 shows the screening results of an artificial intelligence model (our model) that applies a thermodynamic-based acquisition function and the existing methodology (Bare BO).

The horizontal axis represents the number of samples, and the vertical axis represents η_{ows}, S_{config}, C, and I.

C and 1 represent the changes in the hyperparameters of the GPR model.

η_{ows} represents the water electrolysis overpotential, which is the sum of the overpotential required to achieve an oxygen evolution current density of 10 mA cm⁻² and the overpotential required to achieve a hydrogen evolution current density of −10 mA cm⁻². In the Bayesian optimization technique, the acquisition function recommends five compositions for each round. The existing methodology, shown in gray, derived the optimal composition of Co₅₀Ni₄₀Pd₅Pt₅ through 100 sample synthesis cycles, with a water electrolysis overpotential of 455 mV S_{config} below represents the entropy of each composition, and for a catalyst with n elements, the compositional entropy may be calculated using Equation 4:

$${S}_{config} = - {\sum }_{i = 1}^{n} {x}_{i} ln {x}_{i} \qquad [ Equation 4 ]$$

Here, S_{config} represents the compositional entropy, n represents the number of elements, and x represents the composition of the ith element.

Using Equation 4, it is possible to confirm that the optimal composition derived from the existing methodology has a low compositional entropy that does not satisfy the conditions for a high-entropy alloy. This reveals the limitations of existing artificial intelligence models when used to design high-entropy alloys.

Meanwhile, the artificial intelligence model proposed in this invention, shown in blue, shows that the final composition does not fall into a low-entropy local minimum and exhibits a significantly lower overpotential (237 mV) than the comparison group of existing methods.

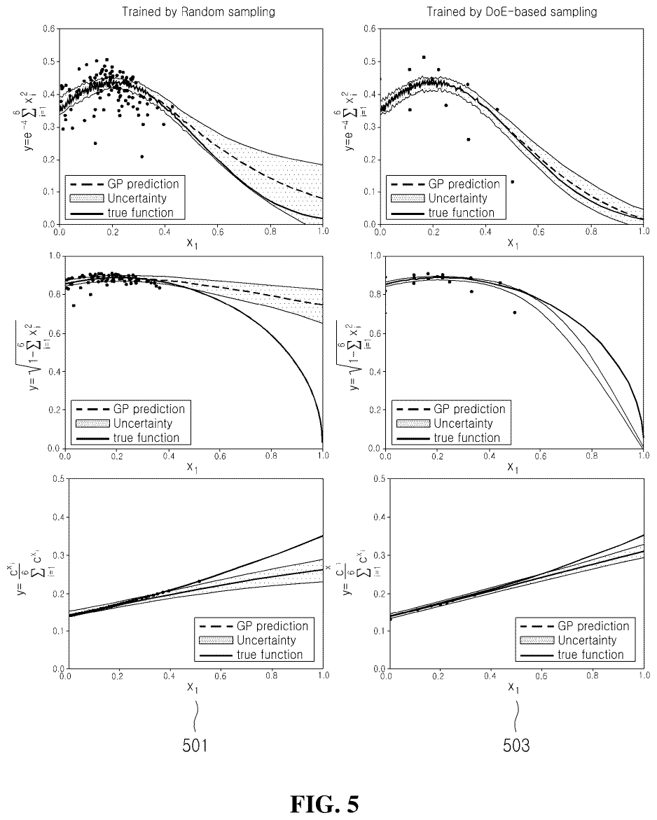

FIG. 5 is a graph illustrating the performance of a design of experiments-based sampling technique according to an embodiment of the disclosure.

The graph shown in FIG. 5 compares the design of experiments-based sampling technique proposed in the disclosure for artificial intelligence model training with the random sampling technique.

The dataset is controlled to have six features (x₁, . . . , x₆) and one parameter (y). The random sampling technique randomly extracts 119 features (x1, . . . , x6) that satisfy the composition conditions, and trains the artificial intelligence model using the y values obtained by substituting these features into the truth function. For example, the truth function may be expressed as shown in Equation 5 below.

$$y = {e}^{- 4 {\sum }_{i = 1}^{6} {x}_{i}^{2}} \qquad [ Equation 5 ]$$

$$y = \sqrt{1 - {\sum }_{i = 1}^{6} {x}_{i}^{2}}$$

$$y = \frac{{e}^{{x}_{i}}}{{\sum }_{i = 1}^{6} {e}^{{x}_{i}}}$$

Here, y represents the truth function, x represents a feature, and i represents the number of features.

In contrast, the design of experiments-based sampling technique considers all possible sub-dimensions and high-entropy regions derived from the six features, extracts 119 features, and trains the artificial intelligence model using the y value obtained by applying these to the truth function.

As shown in the graph (501) in FIG. 5, the artificial intelligence model trained using the random sampling technique shows that the model's predicted values deviate significantly from the actual values as the three truth function modes x1 approach 1. Moreover, the model's uncertainty is also high near where x₁ is 1. This suggests that the sampling process of the existing GPR model overdesigns the uncertainty of edge cases, leading to repeated sampling in low-dimensional regions rather than the desired high-entropy region.

Meanwhile, looking at the graph (503), it shows that the artificial intelligence model trained using a sampling technique based on design of experiments can learn even edge cases, so the artificial intelligence model can predict reality better.

FIG. 6 is a view illustrating a process for designing a catalyst using an artificial intelligence model in a device for designing a catalyst according to an embodiment of the disclosure.

Referring to FIG. 6, the process for designing a catalyst includes: designing experimental conditions through a synthesis process optimization technique (601); designing a composition required for initial learning of an artificial intelligence model based on the designed experimental conditions; selecting a kernel of the artificial intelligence model (605); and designing a final catalyst based on a thermodynamics-based acquisition function.

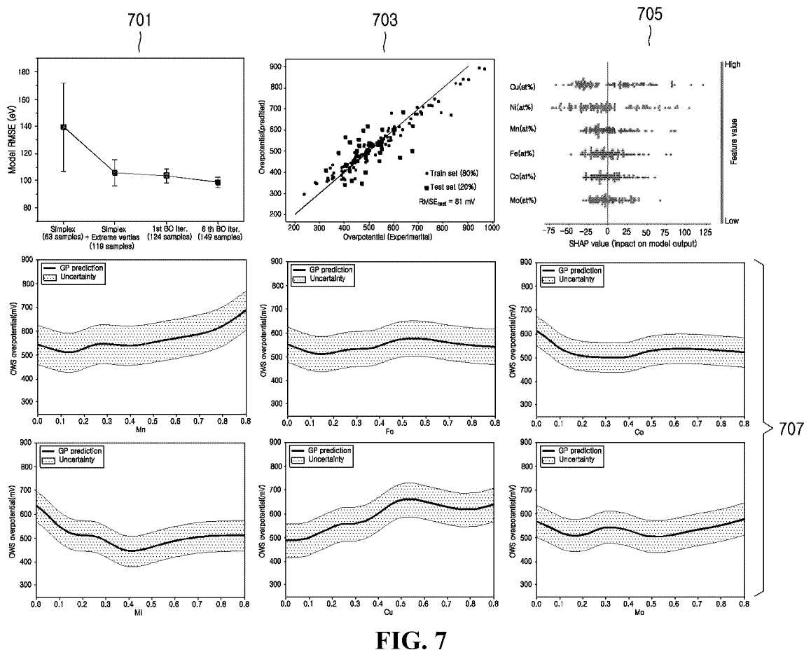

FIG. 7 is a graph illustrating the relationship between overpotential and a high-entropy alloy according to an embodiment of the disclosure.

Looking at FIG. 7, the graph (701) shows the root mean squared error (RMSE) of the artificial intelligence model, and it can be seen that this decreases with each iteration.

The graph (703) depicts the actual overpotential and predicted values after separating the training and test datasets, and the RMSE of the test dataset is 81 mV, demonstrating that it predicts the actual overpotential value fairly well.

The graph (705) shows how each element contributed to the reduction in overpotential. Higher element contents are indicated in red, while lower elements are indicated in blue. It is possible to confirm that, of the six elements initially entered (Mn, Fe, Co, Ni, Cu, Mo), Cu and Mn contribute to increasing overpotential, while Co and Ni significantly reduce overpotential as their contents increase. It is possible to confirm that Mo and Fe have a lower correlation than other elements.

It is possible to confirm that the graph (707) plots the relationship between the composition and overpotential of each element, and for Mn and Cu, increasing the composition contributes to increasing the overpotential. In contrast, Co and Ni contribute to lowering overpotential, but beyond a certain composition, the overpotential tends to increase again. This is because when the composition of one element becomes too high, the resulting product fails to satisfy the conditions for a high-entropy alloy and instead transforms into a mixture of low-entropy alloys.

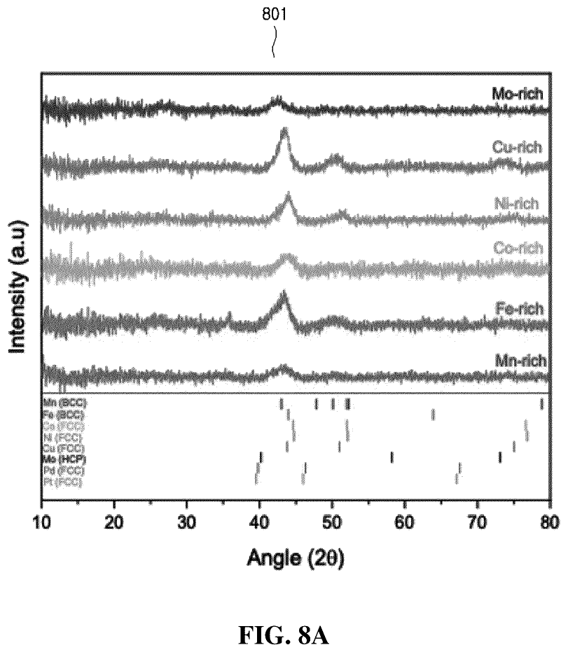

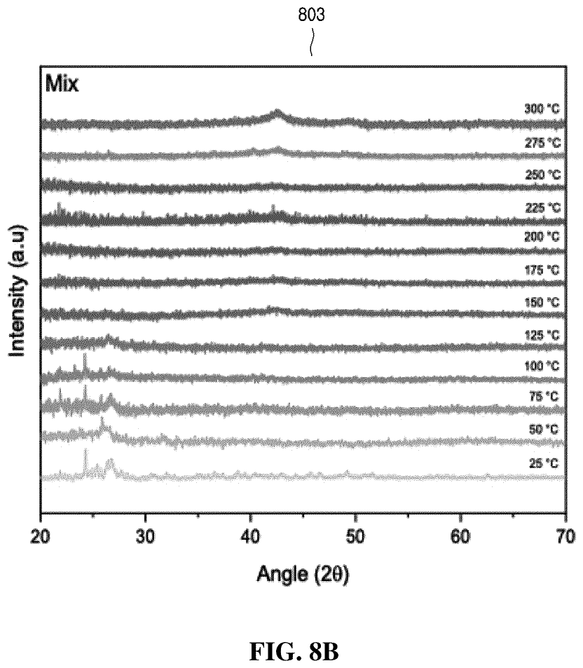

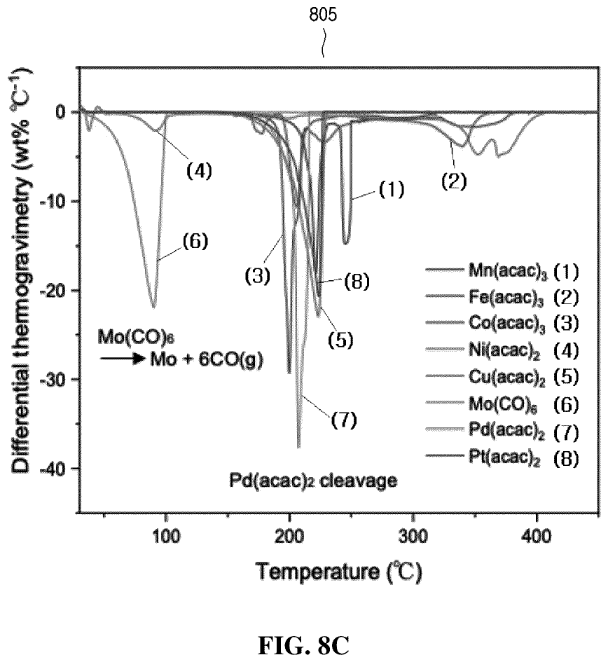

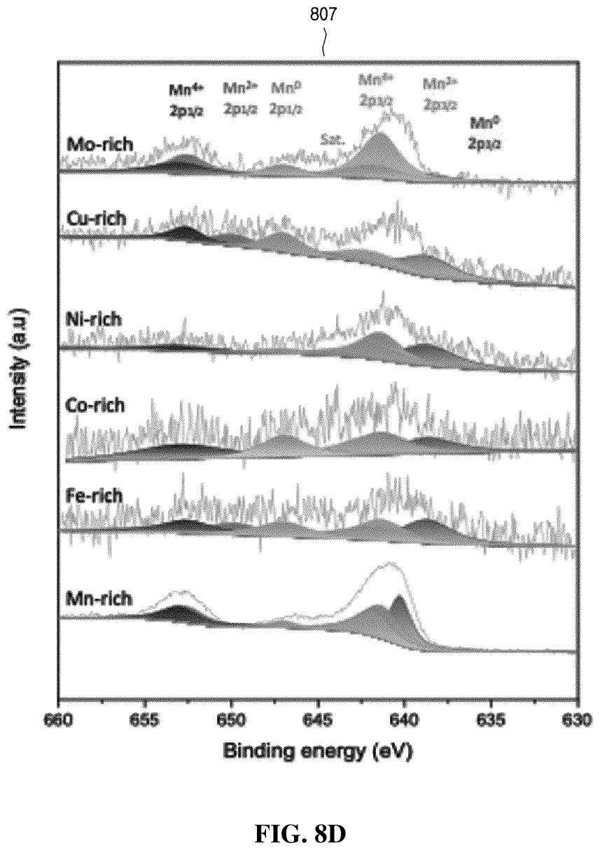

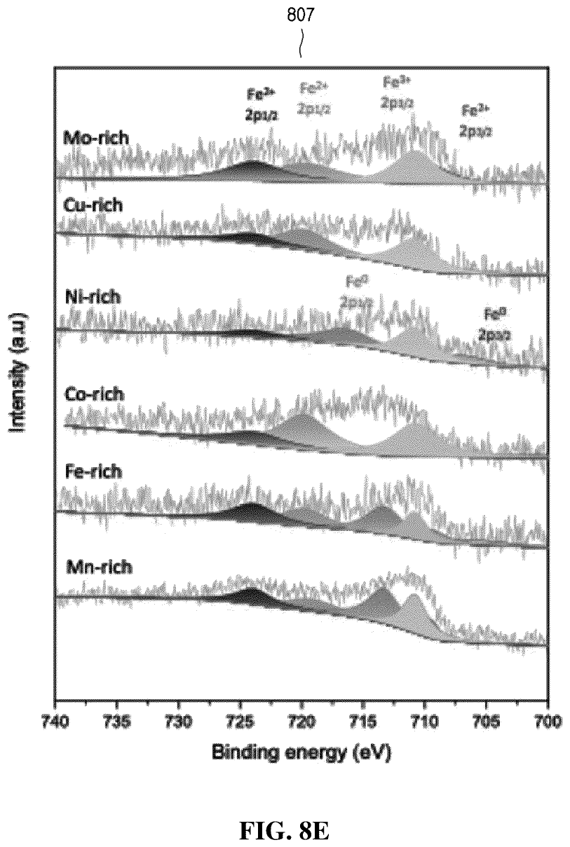

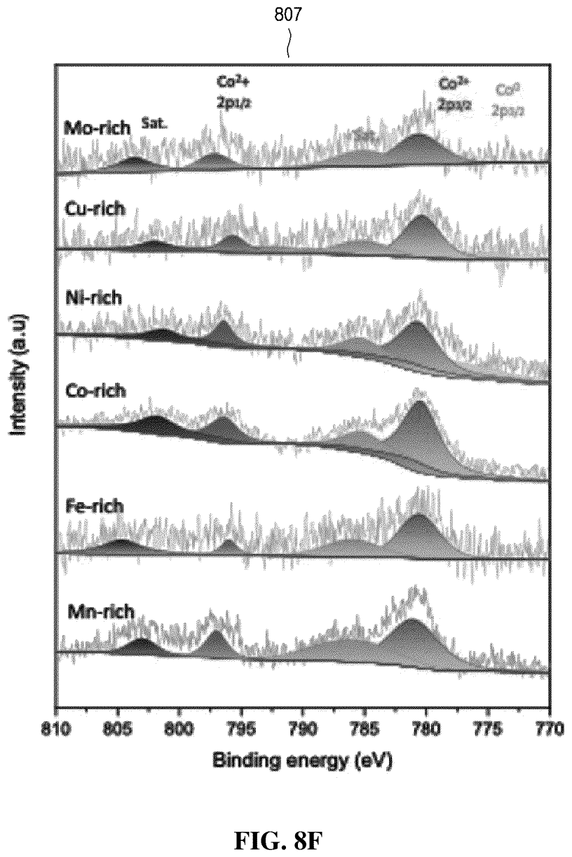

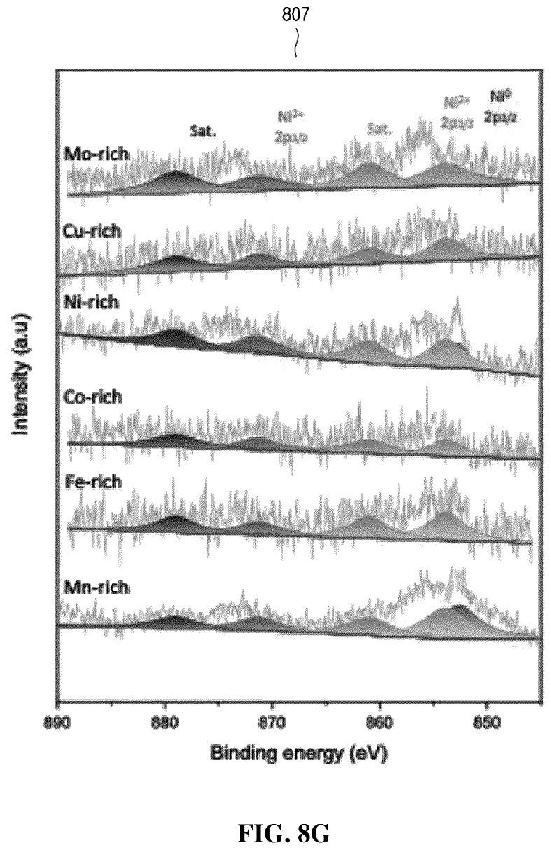

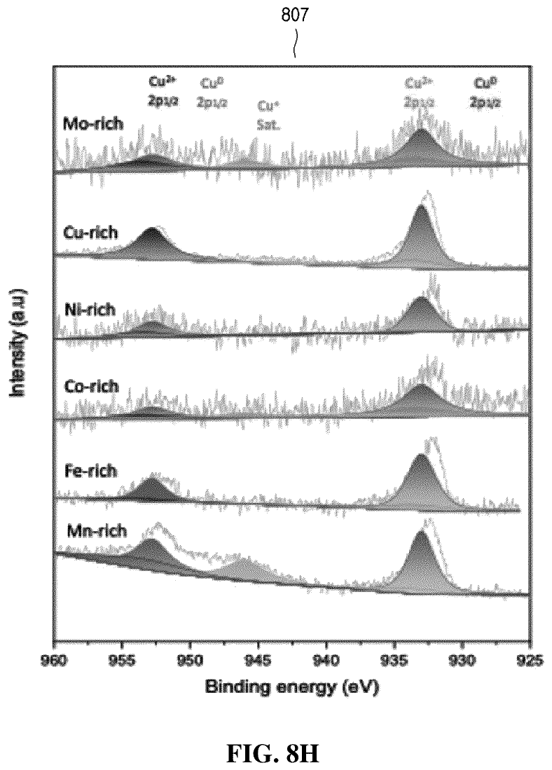

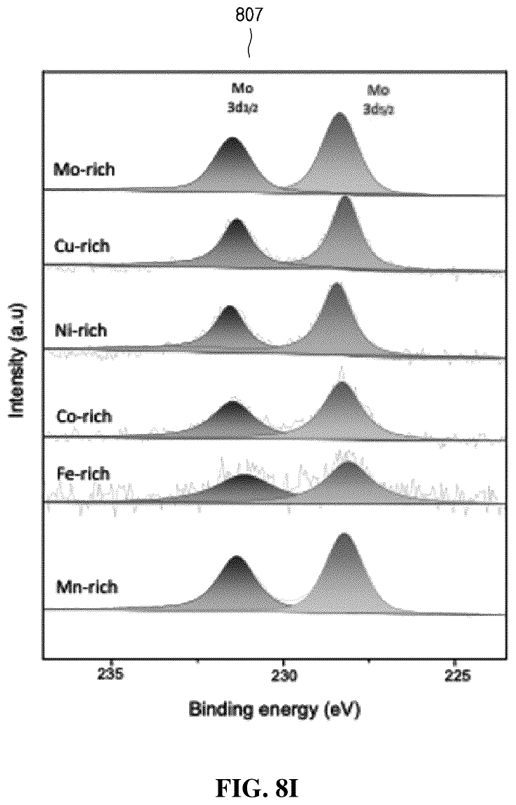

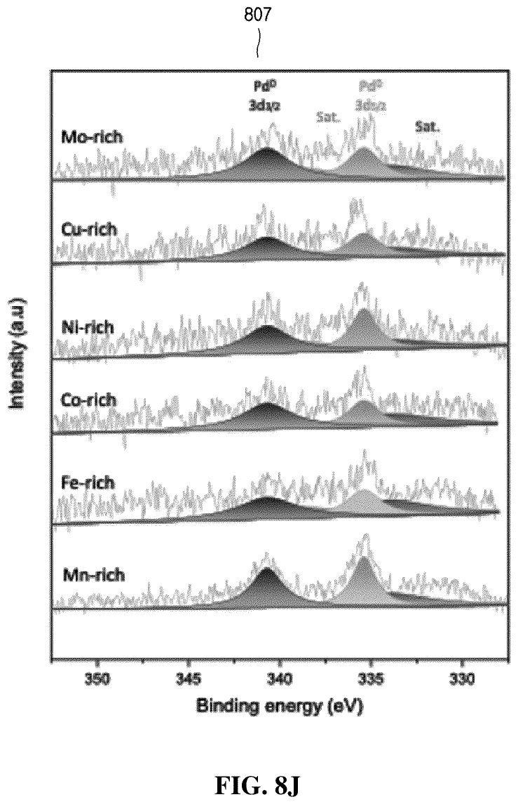

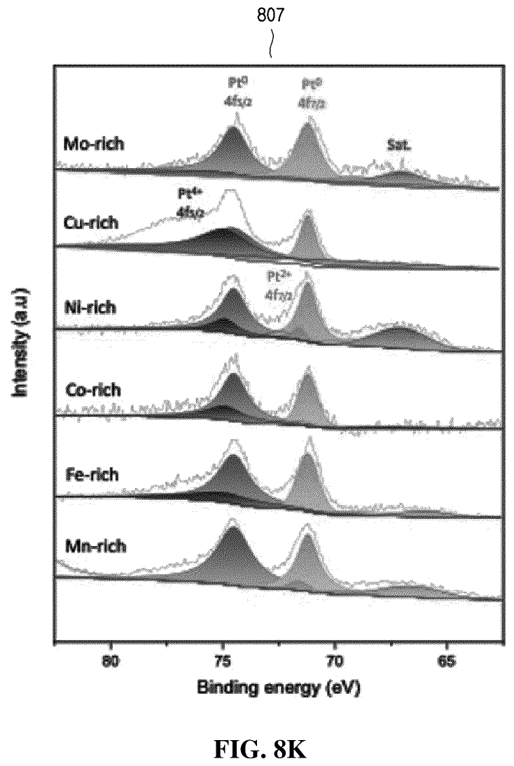

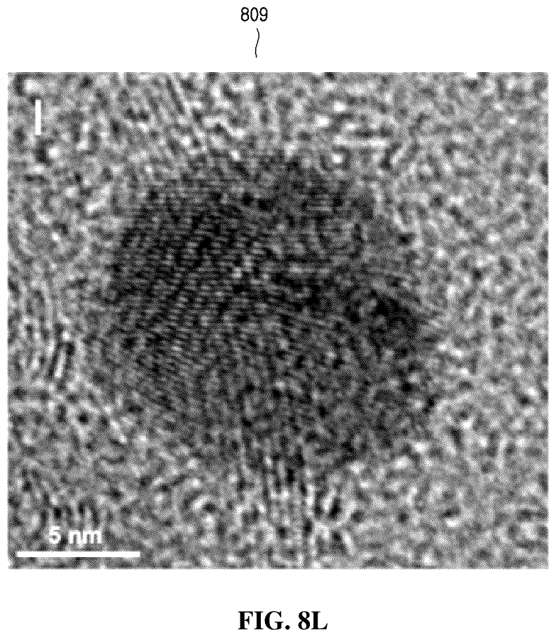

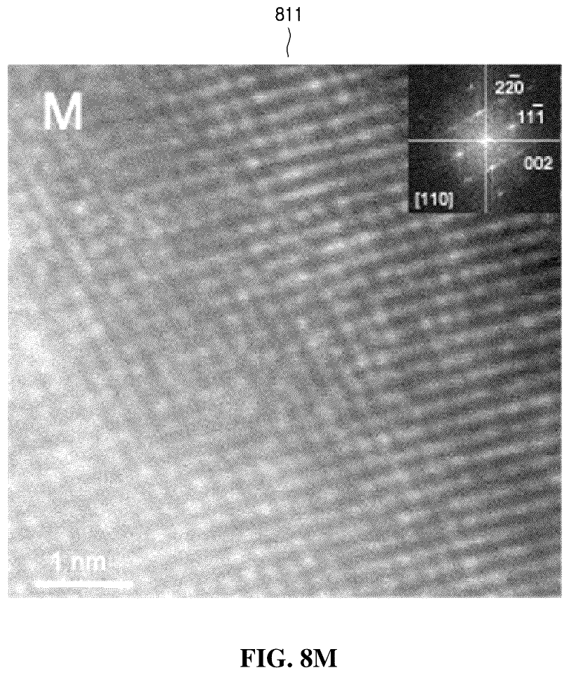

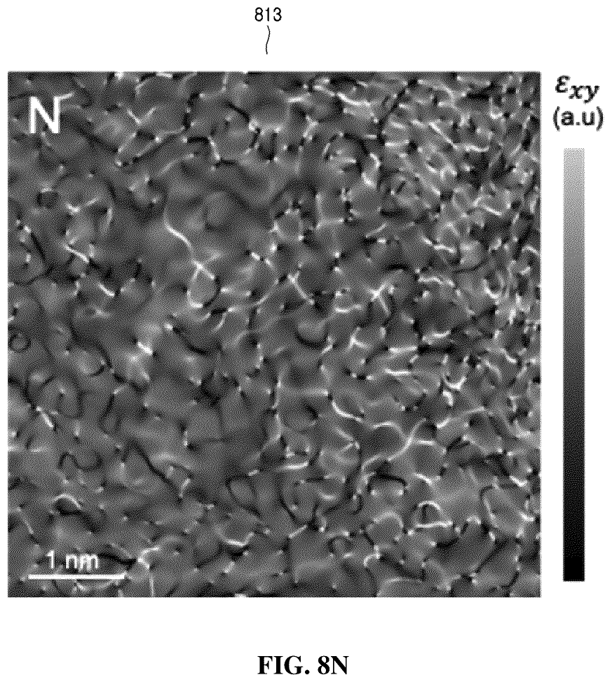

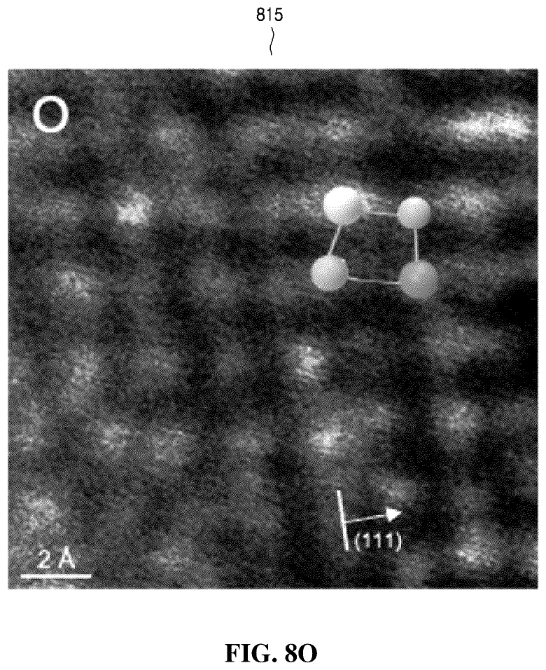

FIG. 8a to 8o are a graph illustrating the structural characteristics of a high-entropy alloy according to an embodiment of the disclosure.

Referring to FIG. 8a to 8o, the graph (801) shows compositions that satisfy the conditions for a high-entropy alloy while also exhibiting a significantly higher concentration of one element, which are designated as Mn-rich, Fe-rich, . . . , and Mo-rich, respectively, and their structural properties are compared using X-ray diffraction (XRD) data. Below, the XRD peak locations for each metal are indicated. The high-entropy alloy peaks do not completely overlap with the positions of the metal pits, confirming that phase separation did not occur in these six samples. Since the other samples have higher configuration entropy than these six, it is possible to indirectly confirm that all samples synthesized using the dispenser are high-entropy alloys.

The graph (803) shows the structural characteristics of the metal precursors (metal acetylacetonates) used for the synthesis of high-entropy alloys, which were uniformly mixed and heated to high temperatures. At around 175-200 °C, where the metal acetylacetonate bonds are broken, the metal precursor peak disappears, and the broad XRD peak characteristic of high-entropy alloys can be observed.

The graph (805) shows the analysis using Differential ThermoGravimetry (DTG), in which a thermogravimetric analysis (TGA) is differentiated with respect to temperature, and Mo(CO)₆ decomposes first at low temperatures, while Pd(acac)₂ exhibits a sharp peak.

The graph (807) shows the results of an X-ray photoelectron spectroscopy (XPS) analysis of the six samples depicted in Graph (801).

The image (809) shows a transmission electron microscope (TEM) image of a high-entropy alloy synthesized with an optimal composition. The image (811) is a TEM image magnified to atomic resolution.

The image (813) shows the TEM image of the image (811) analyzed using geometric phase analysis, indicating the distance of each atom from its ideal position. The image (813) reveals the severe lattice distortion characteristic of a high-entropy alloy. The image (815) also supports this, and it is possible to confirm that the noble metals, which appear as brightly glowing atoms in the scanning transmission electron microscope (STEM) image of the image (815), are not clumped together but are evenly distributed on the surface.

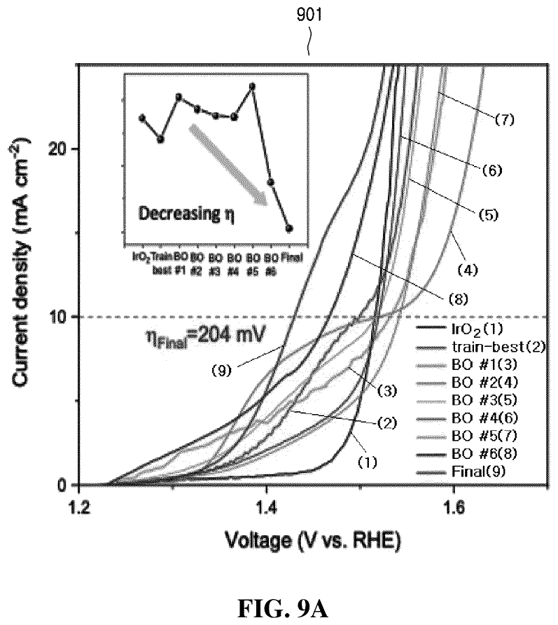

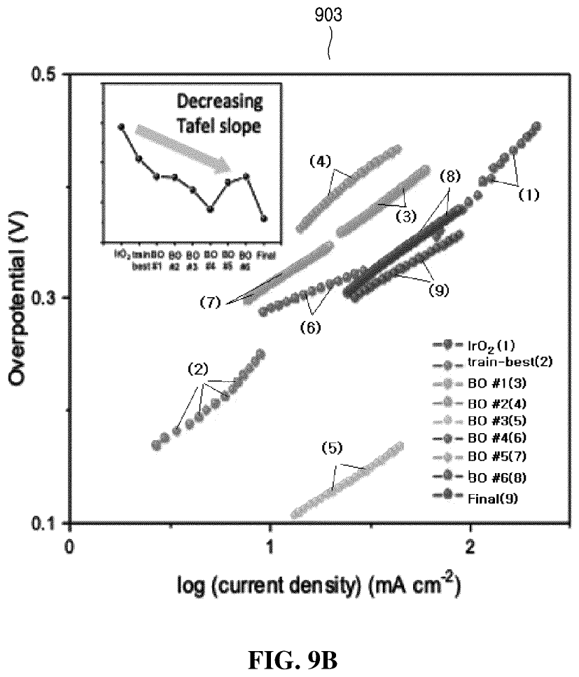

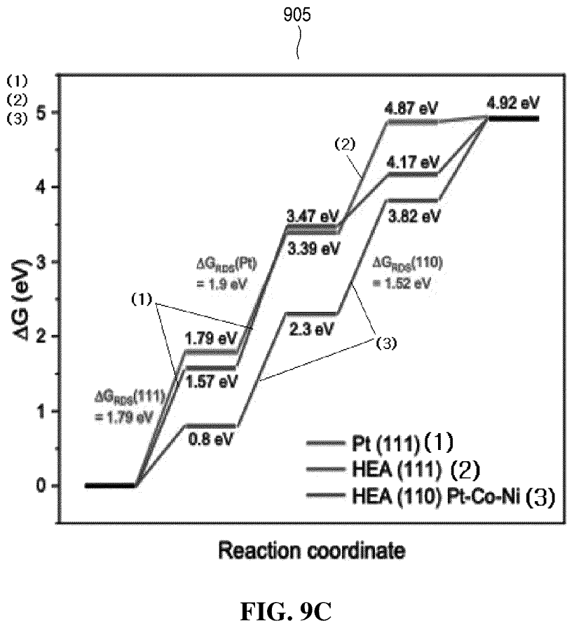

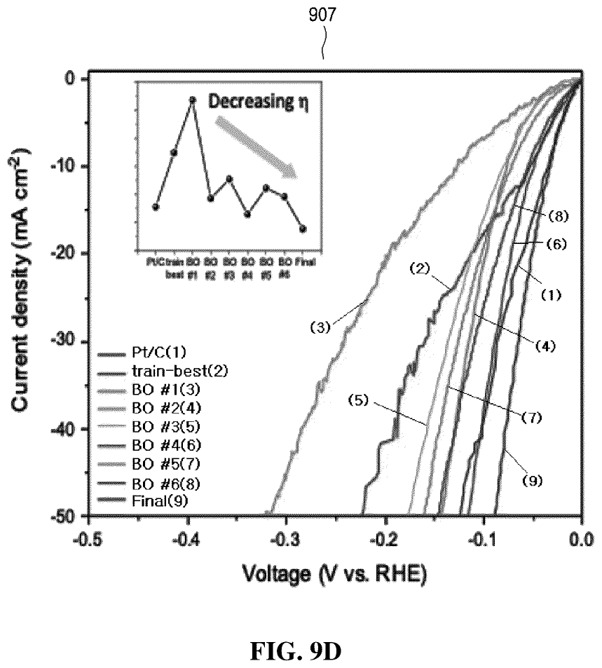

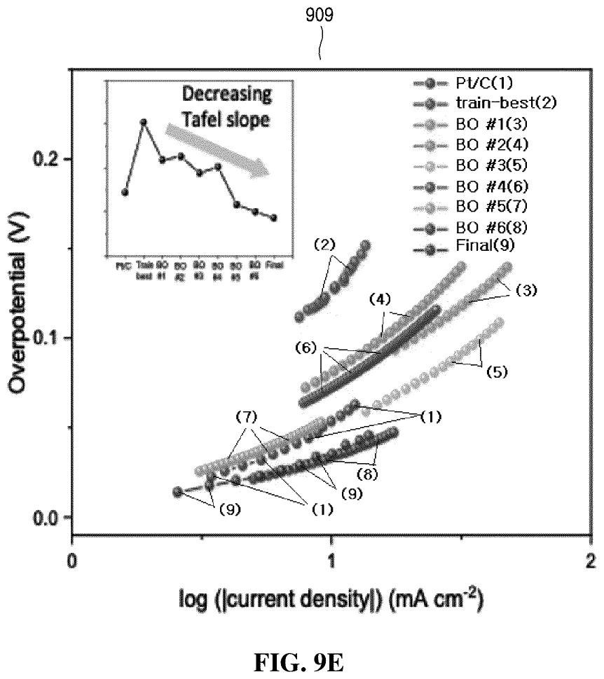

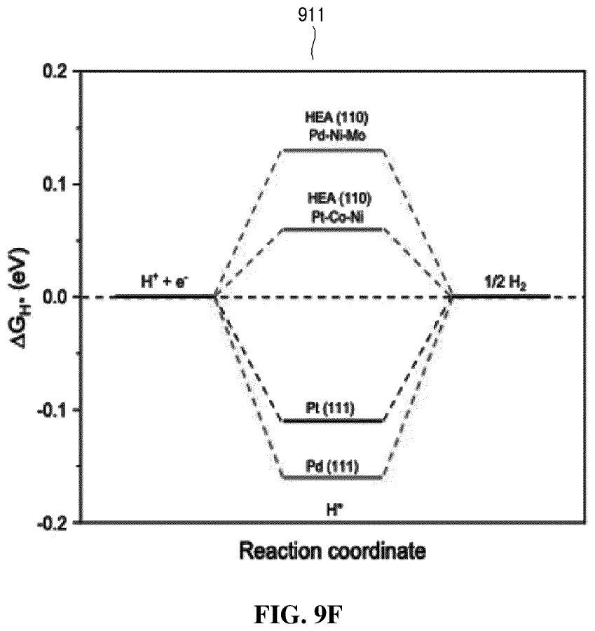

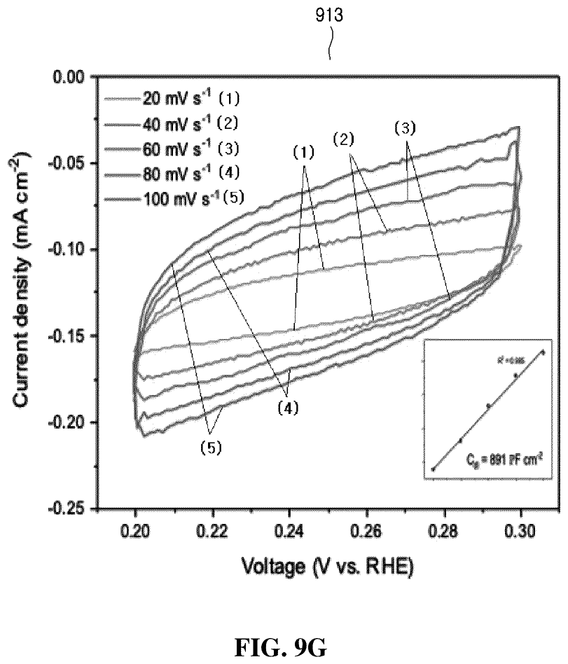

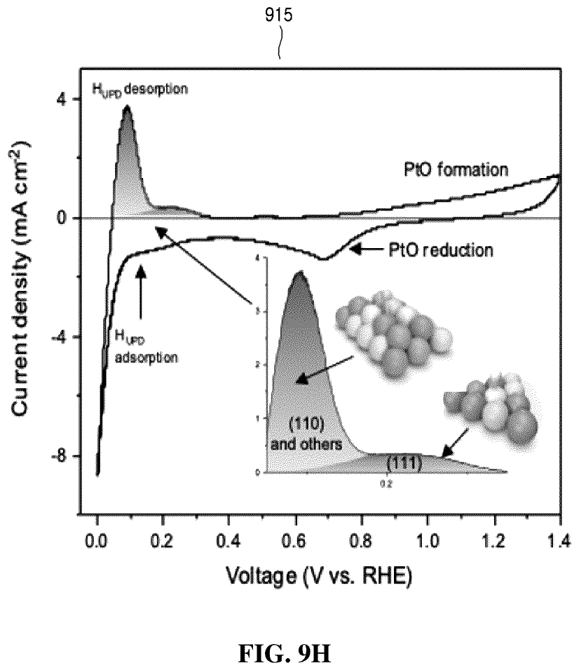

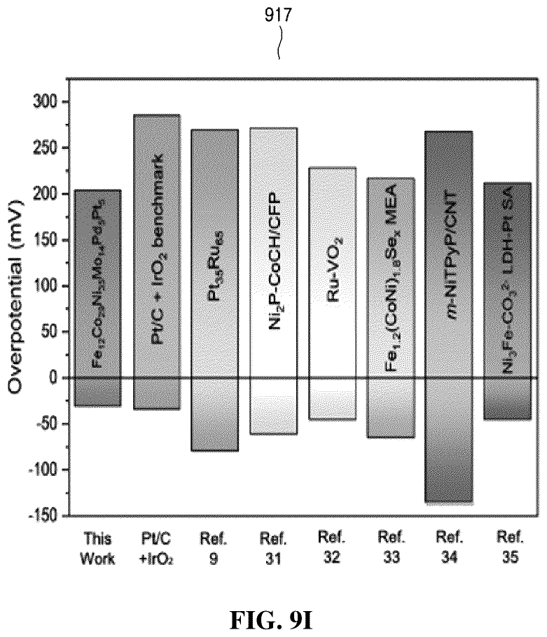

FIG. 9a to 9i are a graph illustrating electrochemical test results of a high-entropy alloy according to an embodiment of the disclosure.

The graphs (901) and (903) represent the LSV and Tafel plots of the OER region, and graph (905) represents the DFT calculation results. The graphs (901, 903, and 905) demonstrate that the overpotential and Tafel slope decrease as Bayesian optimization progresses.

The graphs (907) and (909) represent the LSV and Tafel plots of the HER region, and graph (911) represents the DFT calculation results for the HER region. The graphs (907, 909, and 911) demonstrate that the overpotential and Tafel slope decrease. In graphs (907 and 909), BO #1 through BO #6 represent the best-performing samples among the five recommended for each round of Bayesian optimization.

The graph (913) shows the ECSA of the final sample, and the graph (915) shows the underpotential deposition (UPD) of the (110) plane, calculated as the reaction site. The graph (917) compares the performance with that of existing noble metal catalysts.

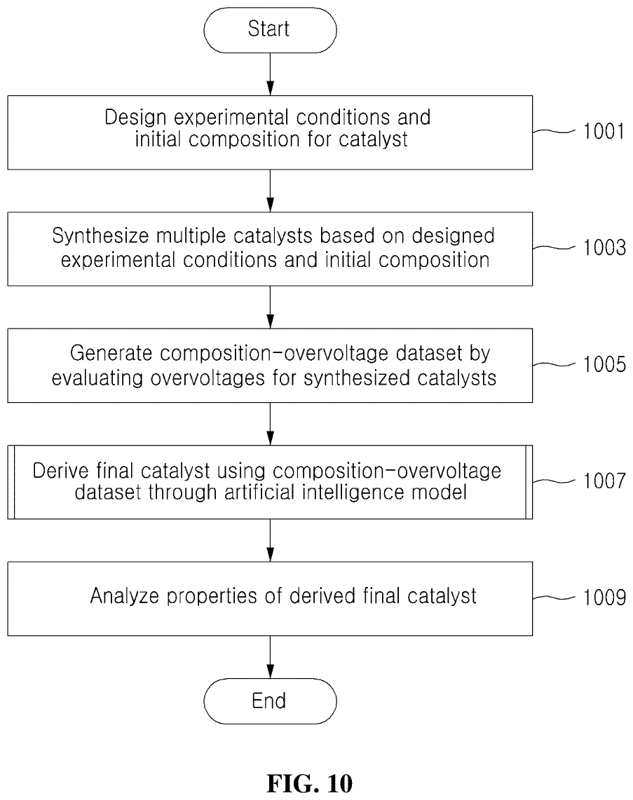

FIG. 10 is a flowchart of designing a catalyst in a device according to an embodiment of the disclosure.

Referring to FIG. 10, the control unit (101) of the catalyst estimation device designs experimental conditions (or synthesis conditions) and initial composition for the catalyst based on a design of experiments in step 1001. At this time, the control unit (101) determines the synthesis conditions according to a synthesis process optimization technique and designs the initial catalyst based on the determined synthesis conditions.

For example, the synthesis process optimization technique (601) may be controlled by five synthesis conditions (e.g., metal precursor concentration, solvent, additive, reaction time, and reaction temperature), as illustrated in FIG. 6. When each of the synthetic conditions is represented by the symbols A, B, C, D, and E, A may represent oleylamine (OAm) versus ethanol as a solvent condition, B may represent carbon nanotubes (CNTs) versus no CNT as an additive, C may represent 200 °C versus 120 °C as a reaction temperature, D may represent 12 hours versus 2 hours as a reaction time, and E may represent 5 mM versus 10 mM as a metal precursor concentration.

The synthesis process optimization technique (601) may employ a writing scheme where a symbol is indicated when a particular synthesis condition corresponds to the former, and no symbol is indicated when a particular synthesis condition corresponds to the latter. For example, the combination “ACD” may mean the combination of (OAm, CNT-free, 200 °C, 12 hours, 10 mM).

In this way, if there is a negative correlation between two options for each synthesis condition, the total number of possible synthetic scenarios (32) may be organized into two groups of 16. For example, it can be expressed as I(ethanol, no CNT used, 120 °C, 2 hours, 10 mM)=−ABCDE, A=−BCDE, B=−ACDE, . . . , DE=−ABC, etc. Using this organization method, the synthesis process optimization technique (601) may optimize the synthesis process by selecting the highest-performing synthesis process within 16 experiments for five conditions. For example, the optimized synthesis process may include the synthesis conditions: (OAm solvent, CNT use, 200 °C heat treatment, 2 hours of heat treatment, and 5 mM precursor concentration).

The control unit (101) then designs a catalyst based on the optimized synthesis process and designs the initial composition of the resulting catalyst.

In step 1003, the control unit (101) automatically synthesizes multiple catalysts based on the designed experimental conditions and initial compositions through the dispenser. At this time, the control unit (101) may synthesize multiple catalysts with identical constituent elements but different compositions using a sampling technique based on a design of experiments.

For example, the control unit (101) may automatically synthesize samples corresponding to 119 compositions based on 119 points selected through the dispenser.

In step 1005, the control unit (101) evaluates the overpotentials corresponding to the synthesized samples to generate a composition-overpotential dataset. For example, the control unit (101) may measure the overpotentials corresponding to the synthesized samples and convert the measured overpotentials corresponding to the synthesized samples into data to generate a composition-overpotential dataset.

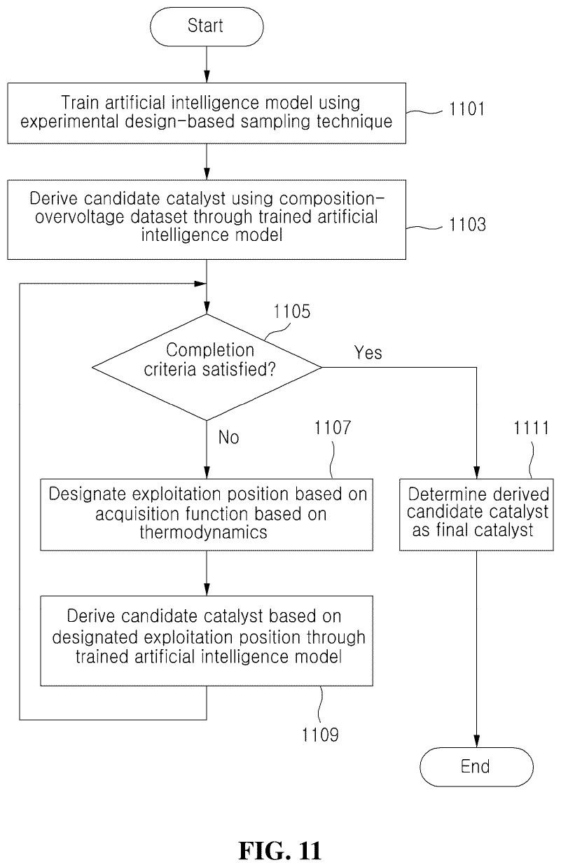

In step 1007, the control unit (101) derives the final catalyst using the composition-overpotential dataset via an artificial intelligence model. The process of deriving the final catalyst will now be described with reference to FIG. 11.

FIG. 11 is a flowchart of searching for a final catalyst using artificial intelligence in a device according to an embodiment of the disclosure.

Referring to FIG. 11, the control unit (101) of the catalyst estimation device trains and tests an artificial intelligence model in step 1101 using a sampling technique based on a design of experiments. For example, the control unit (101) may classify 80% of the 119 data sets included in the composition-overpotential dataset as a training dataset and 20% as a test dataset. Then, based on the training and test datasets classified by the artificial intelligence model, the control unit (101) may derive results for multiple kernels, and among the results derived from the multiple kernels, the kernel with the best performance may be selected as the main kernel constituting the artificial intelligence model.

In step 1103, the control unit (101) derives candidate catalysts using the composition-overpotential dataset through the trained artificial intelligence model.

In step 1105, the control unit (101) checks whether the derived candidate catalyst meets a predefined completion criterion. For example, the completion criterion represents the criteria for completing the operation of deriving a catalyst using the trained artificial intelligence model and may include a pre-specified number of convergences for the trained artificial intelligence model.

As a result of the check, if the derived candidate catalyst meets a completion criterion, the control unit (101) proceeds to step 1111. Otherwise, the control unit (101) proceeds to step 1107.

If step 1111 is performed, the control unit (101) determines the derived candidate catalyst as the final catalyst.

Conversely, if step 1107 is performed, the control unit (101) uses an acquisition function based on thermodynamic entropy to designate a development location for the trained artificial intelligence model.

For example, the control unit (101) may generate an acquisition function to create an artificial intelligence model that not only finds the lowest overpotential but also has confidence (low uncertainty) in all regions of the configuration space. Furthermore, the control unit (101) may use the generated acquisition function to search for data points for the artificial intelligence model to learn additionally and designate the searched data points as the next search location.

For example, the acquisition function may be a function that indicates that, because the artificial intelligence model does not know about thermodynamic entropy and composition conditions, the amount of information obtained from sampling edge cases is less than the amount of information obtained in high-entropy regions.

For example, the composition conditions may be expressed as in Equation 1. For example, the acquisition function may be designed using Equation 2. For example, once the acquisition function is designed, the optimal x value may be obtained using Equation 3.

In step 1109, the control unit (101) derives candidate catalysts based on the designated pioneering locations using the trained artificial intelligence model, and then proceeds to step 1105. For example, the trained artificial intelligence model may perform learning based on the designated exploration locations to derive candidate catalysts.

Returning to FIG. 10, the control unit (101) analyzes the properties of the derived final catalyst in step 1009. For example, the control unit (101) may measure the electrochemical properties of the final catalyst using a measuring instrument and store the measured values.

Thereafter, at the user's request, the control unit (101) may display the experimental conditions, composition, and stored electrochemical properties of the final catalyst through the display unit (105). For example, the control unit (101) may display at least one of the experimental conditions and electrochemical properties simultaneously with the composition of the final catalyst.

Through this process, an embodiment of the disclosure may accurately design a catalyst that satisfies the high-entropy criterion using artificial intelligence. An embodiment of the disclosure may rapidly design a high-entropy alloy as a catalyst using AI.

The description of the disclosure is for illustrative purposes, and those skilled in the art will understand that it can be easily modified into other specific forms without changing the technical idea or essential features of the disclosure. Therefore, the embodiments described above should be understood as being exemplary in all respects and not limiting. For example, each component described as a single type may be implemented in a distributed manner, and likewise, components described as distributed may be implemented in a combined form.

The scope of the disclosure is indicated by the following claims, and all changes or modifications derived from the meaning and scope of the claims and their equivalent concepts should be interpreted as being included in the scope of the disclosure.

### EXPLANATION OF REFERENCE NUMERALS

101: control unit
103: memory
105: display unit
107: Input/output unit
201: experimental conditions and initial composition design unit
203: catalyst synthesis unit
205: artificial intelligence model unit
207: property analysis unit

What is claimed is:

1. A device for designing a catalyst using artificial intelligence, the device comprising:
a dispenser;
a measuring instrument; and
a control unit configured to design experimental conditions and an initial composition for the catalyst, synthesize multiple catalysts based on the experimental conditions and the initial composition through the dispenser, evaluate multiple overpotentials corresponding to the multiple catalysts to generate a composition-overpotential dataset, derive a final catalyst using the composition-overpotential dataset through an artificial intelligence model, and analyze physical properties of the final catalyst through the measuring instrument.

2. The device of claim 1, wherein the artificial intelligence model is a Gaussian process regression model.

3. The device of claim 1, wherein the final catalyst is a high-entropy alloy.

4. The device of claim 1, wherein the control unit is configured to train the artificial intelligence model using a sampling technique based on a design of experiments, derive a candidate catalyst using the composition-overpotential dataset through the trained artificial intelligence model, check whether the candidate catalyst satisfies a predetermined completion criterion, and, if the candidate catalyst does not satisfy the predetermined completion criterion as a result of checking, designate a pioneering position using an acquisition function based on thermodynamics, and perform learning based on the pioneering position through the trained artificial intelligence model to derive a new candidate catalyst.

5. The device of claim 4, wherein the control unit is configured to determine the candidate catalyst as the final catalyst if, as a result of checking, the candidate catalyst satisfies the predetermined completion criterion.

6. A method for designing a catalyst using artificial intelligence, the method comprising:
designing, by a control unit, experimental conditions and an initial composition for the catalyst;
synthesizing, by the control unit, multiple catalysts based on the experimental conditions and the initial composition through a dispenser;
evaluating, by the control unit, multiple overpotentials corresponding to the multiple catalysts to generate a composition-overpotential dataset;
deriving, by the control unit, a final catalyst using the composition-overpotential dataset through an artificial intelligence model; and
analyzing, by the control unit, physical properties of the final catalyst through a measuring instrument.

7. The method of claim 6, wherein the artificial intelligence model is a Gaussian process regression model.

8. The method of claim 6, wherein the final catalyst is a high-entropy alloy.

9. The method of claim 6, wherein the deriving the final catalyst comprises:
training the artificial intelligence model using a sampling technique based on a design of experiments;
deriving a candidate catalyst using the composition-overpotential dataset through a trained artificial intelligence model;
checking whether the candidate catalyst satisfies a predetermined completion criterion;
if the candidate catalyst does not satisfy the predetermined completion criterion as a result of checking, designating a pioneering position using an acquisition function based on thermodynamics; and
performing learning based on the pioneering position through the trained artificial intelligence model to derive a new candidate catalyst.

10. The method of claim 9, further comprising determining the candidate catalyst as the final catalyst if, as a result of checking, the candidate catalyst satisfies the predetermined completion criterion.
